(19) United States
(12) Patent Application Publication
(10) Pub. No.: US 2013/0144243 A1
(43) Pub. Date: Jun. 6, 2013

(54) Absorbent Product

(71) Applicants: Hirofumi Miyake, Tokushima (JP); Akiko Tatsukawa, Tokushima (JP)

(72) Inventors: Hirofumi Miyake, Tokushima (JP); Akiko Tatsukawa, Tokushima (JP)

(73) Assignee: LIVEDO CORPORATION, Ehime (JP)

(21) Appl. No.: 13/639,291

(22) PCT Filed: Apr. 13, 2011
(86) PCT No.: PCT/JP2011/002190

§ 371 Date: Oct. 4, 2012

(30) Foreign Application Priority Data
Apr. 16, 2010 (JP) 2010-095143

(51) Int. Cl. A61F 13/495

(52) U.S. Cl. CPC A61F 13/495
USPC 604/385.19

(57) ABSTRACT

In an absorbent product, a front pocket, a pair of side pockets and a rear pocket are formed between a back sheet member, and a front absorbent core, a pair of side absorbent cores and a rear absorbent core, and excrement which has passed through an opening is collected therein. In the absorbent product, the side absorbent cores are bonded on inner surfaces of standing gather parts. Thus, since the standing gather parts stand up, the side absorbent cores become far away from the back sheet member. Side wall parts of the side pockets have structure where the side absorbent cores are bonded on the inner surfaces of side sheets and outer covering sheet. Thus, it is possible to increase stiffness of the side wall parts of the side pockets, to thereby suppress deformation of the side wall parts due to a weight of excrement collected in the side pockets.

### TECHNICAL FIELD

The present invention relates to an absorbent product for receiving excrement from a wearer.

### BACKGROUND ART

In an absorbent product such as a disposable diaper, various proposals are conventionally made in order to prevent loose stool from attaching over a large area of skin of a wearer by spreading through a gap between the wearer and the absorbent product, when the loose stool containing much moisture is excreted.

For example, in a disposable diaper of Japanese Patent Application Laid-Open No. 2002-209941 (Document 1), a second core is provided on a front part of a first core extending across almost an entire length thereof, and a space between the first core and the second core is a pocket to collect excrement. In the disposable diaper of Document 1, a front end of the second core is fixed on the first core, intersect portions between both side portions in the second core and a free end portion lying near the crotch area are connected to free edge portions of leakage prevention cuffs. Since the leakage prevention cuffs stand up, the free end portion of the second core is away from the first core to form an opening of the pocket.

In the disposable diaper of Document 1, since each side wall part of the pocket is constructed with the leakage prevention cuff formed of thin nonwoven fabric, stiffness of the side wall part of the pocket is not enough and a weight of stool collected in the pocket cannot be supported. Thus, standing of the leakage prevention cuff is inhibited by the weight of stool and leakage of urine or the like from side portions of the disposable diaper (so-called lateral leakage) possibly occurs.

### SUMMARY OF INVENTION

The present invention is intended for an absorbent product for receiving excrement from a wearer. It is an object of the present invention to increase stiffness of side wall parts of side pockets, to thereby suppress deformation of the side wall parts due to a weight of excrement.

The absorbent product according to the present invention comprises: a back sheet member; an absorbent sheet member which is laid on the back sheet member and whose outer edge portion is bonded on the back sheet member, the absorbent sheet member having an opening at a position to face a crotch region of a wearer, the back sheet member being exposed through the opening; and a pair of standing gather parts lying on left and right side portions of the absorbent sheet member and extending in a longitudinal direction, a pair of standing gather elastic members along the longitudinal direction being bonded to the pair of standing gather parts, respectively; wherein the absorbent sheet member comprises: a first absorbent core located at a position to face one of a front side and a rear side of the wearer; a pair of second absorbent cores located at both sides of the opening in a left-right direction; and a liquid-pervious core covering sheet which covers the first absorbent core and the pair of second absorbent cores; a main pocket elongating in the longitudinal direction from the opening is formed between the first absorbent core and the back sheet member, a pair of side pockets deepening in the left-right direction from the opening is formed between the pair of second absorbent cores and the back sheet member, and the pair of second absorbent cores is bonded to the pair of standing gather parts through the core covering sheet.

In the present invention, it is possible to increase stiffness of side wall parts of the side pockets, to thereby suppress deformation of the side wall parts due to a weight of excrement.

According to a preferred embodiment of the present invention, the absorbent sheet member further comprises a pair of second absorbent core elastic members which is located along the longitudinal direction on surfaces, facing the back sheet member, of the pair of second absorbent cores, the pair of second absorbent core elastic members exerting a contractive force on the pair of second absorbent cores. Therefore, the second absorbent cores can be further away from the back sheet member.

According to another preferred embodiment of the present invention, free edges of the pair of standing gather parts are away from bonding areas between the pair of standing gather parts and the pair of second absorbent cores, the pair of standing gather elastic members is located between the free edges and the bonding areas in the pair of standing gather parts, and another pair of standing gather elastic members is bonded to the pair of standing gather parts while overlapping with the bonding areas along the longitudinal direction. Therefore, the stiffness of the side wall parts of the side pockets can be further increased to further suppress deformation of the side wall parts.

According to an aspect of the present invention, a core edge, which lies in the vicinity of the opening or contacts with the opening, of the first absorbent core has a concave portion depressed in the longitudinal direction at the middle in the left-right direction. Therefore, while it is ensured that a length of the opening in the longitudinal direction is large, lengths, in the longitudinal direction, of the side wall parts of the side pockets can be shortened, and as the result, the stiffness of the side wall parts can be further increased.

According to another aspect of the present invention, the absorbent sheet member further comprises a first absorbent core elastic member which is bonded along the longitudinal direction to the first absorbent core at a middle area in the left-right direction, the first absorbent core elastic member exerting a contractive force on the first absorbent core. Thus, the middle area of the first absorbent core becomes far away from the back sheet member. In this case, preferably, the first absorbent core has a weakening line which overlaps with the first absorbent core elastic member at the middle area. Therefore, the first absorbent core easily deforms into a form protruding toward the wearer and collective capacity of the main pocket can be increased.

According to still another aspect of the present invention, the first absorbent core and the pair of second absorbent cores are continuous with each other in the longitudinal direction and formed as one unit. Thus, when the second absorbent cores become far away from the back sheet member by rising of the standing gather parts, a portion of the first absorbent core which lies in the vicinity of the opening can become also far away from the back sheet member.

A preferable absorbent product of the present invention is an auxiliary absorbent pad to be attached on the inside of an exterior product worn by a wearer.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a plan view of an absorbent product.

FIG. 2 is a cross-sectional view of the absorbent product.

FIG. 3 is a cross-sectional view of the absorbent product.

FIG. 4 is a cross-sectional view of the absorbent product.

### DESCRIPTION OF EMBODIMENTS

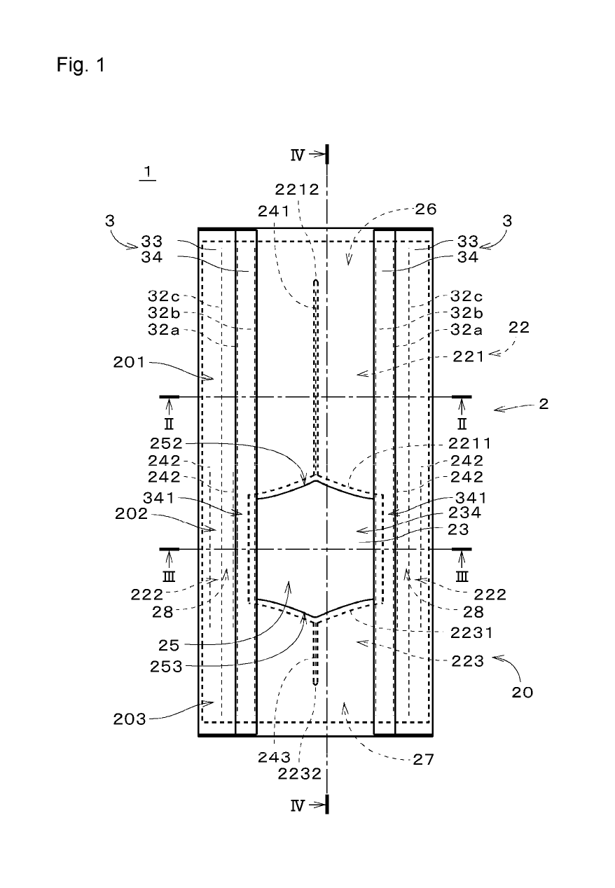

FIG. 1 is a plan view of an absorbent product 1 in accordance with a preferred embodiment of the present invention, and in FIG. 1 the absorbent product 1 is developed in a longitudinal direction (i.e., an up-down direction in FIG. 1). The absorbent product 1 is an auxiliary absorbent pad attached at the inside of a disposable diaper or the like which is an exterior product worn by a wearer, and it receives excrement such as loose stool, from the wearer. FIG. 1 shows a surface of the absorbent product 1 which is to come into contact with the wearer (i.e., the surface is the wearer's side surface).

As shown in FIG. 1, the absorbent product 1 has a sheet-like main body part 2 which has an approximately rectangular shape in a planar view, and a pair of side sheets 3 which is located on both side portions of the main body part 2 in a left-right direction orthogonal to the longitudinal direction (i.e., the left-right direction is a width direction of the main body part 2 and that is identical to the left-right direction in the FIG. 1) and which extends across almost the entire length of the main body part 2 in the longitudinal direction.

An upper portion 201 of the main body part 2 in FIG. 1 is to be positioned on (skin of) the front side (stomach side) of the wearer, and a lower portion 203 in FIG. 1 is to be positioned on the rear side (back side) of the wearer. In the following description, the upper portion 201 and the lower portion 203 are referred to as a “front part 201” and a “rear part 203”, respectively, and a portion 202 to face the crotch region of the wearer at a position between the front part 201 and the rear part 203 is referred to as a “middle part 202”. The middle part 202 is continuous with the front part 201 and the rear part 203.

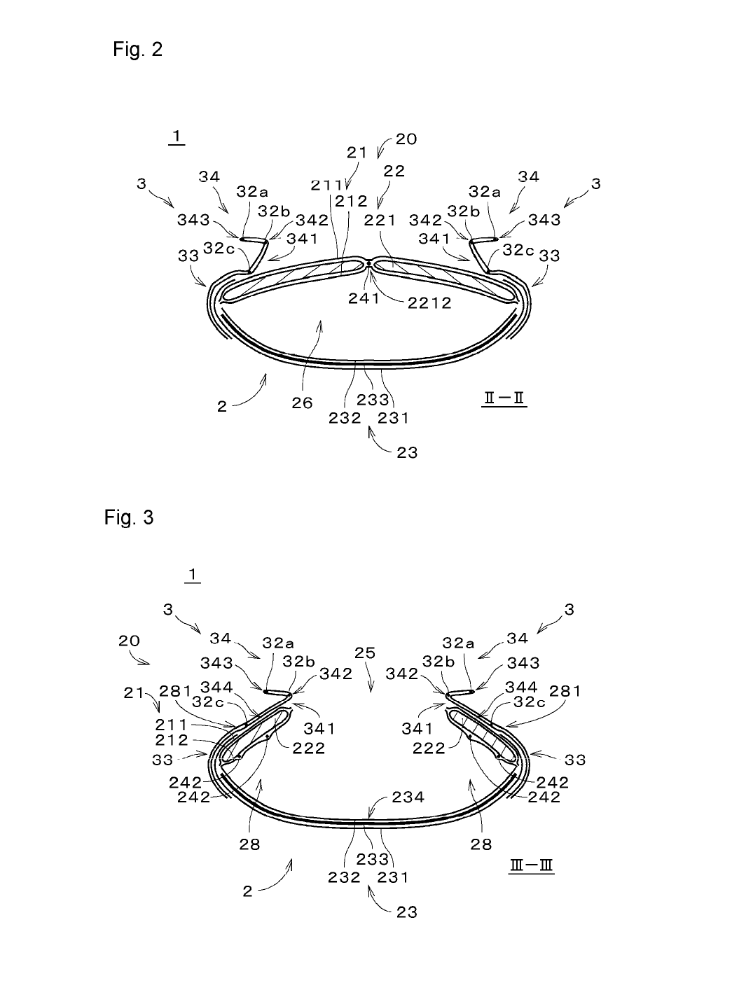

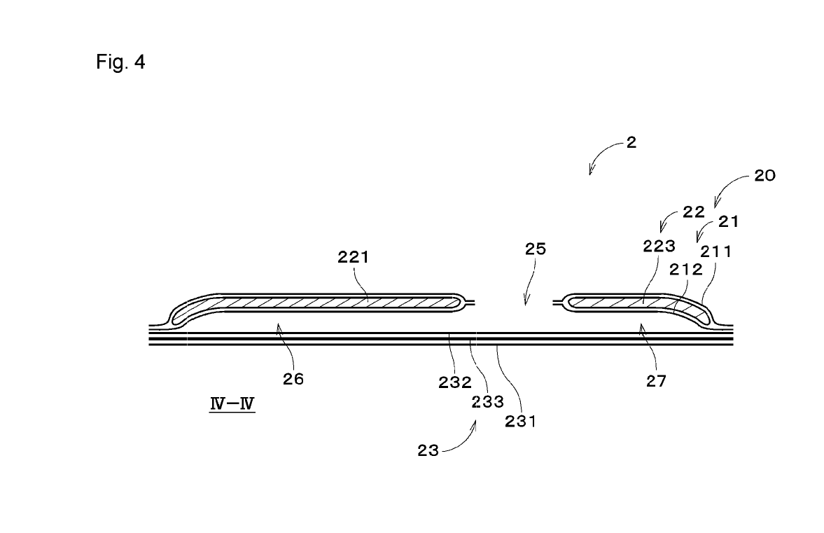

FIGS. 2 and 3 are cross-sectional views of the absorbent product 1 taken along a line II-II and a line III-III in FIG. 1, respectively, where the absorbent product 1 is cross-sectioned with planes orthogonal to the longitudinal direction (i.e., the up-down direction in FIG. 1). FIG. 4 is a cross-sectional view of the absorbent product 1 taken along a line IV-IV in FIG. 1 where the absorbent product 1 is cross-sectioned with a plane orthogonal to the left-right direction. In the absorbent product 1, the rear part 203 has almost same structure as the front part 201 and a cross-sectional view of the rear part 203 cross-sectioned with a plane orthogonal to the longitudinal direction is same as the cross-sectional view shown in FIG. 2.

As shown in FIGS. 1 to 3, each of the pair of side sheets 3 has a strip-like fixed part 33 which is bonded to the left or right side portion of the main body part 2, a gather part 34 which is continuous with the fixed part 33 and in which at least one portion is standing, and a plurality of standing gather elastic members 32a to 32c which are bonded to the gather part 34 along the longitudinal direction. The side sheet 3 is formed by folding a water-repellent or liquid-impervious nonwoven fabric (for example, a spunbond nonwoven fabric, meltblown nonwoven fabric or SMS (spunbond-meltblown-spunbond) nonwoven fabric), which is made of hydrophobic fibers (e.g., polypropylene, polyethylene, polyester, polyamide or nylon), into two portions at a folding line extending in the longitudinal direction, and by locating (sandwiching in) the standing gather elastic members 32a to 32c between the two portions overlapped with each other. For example, a polyurethane yarn, strip-like polyurethane film, yarn-like or strip-like natural rubber, or the like is used as each of the standing gather elastic members 32a to 32c, and in the present embodiment, a polyurethane yarn is used.

Both end portions of the gather part 34 in the longitudinal direction are bonded on the main body part 2. In the absorbent product 1, by contraction of the standing gather elastic members 32a to 32c, a portion 341 of the gather part 34 in which the both end portions in the longitudinal direction are not included (hereinafter the portion 341 is referred to as a “standing gather part 341”) stands up toward the wearer at the side portion of the main body part 2 as shown in FIGS. 2 and 3, so that standing gathers (three-dimensional gathers) which contact with the vicinity of wearer's groin, when worn, are formed. Since the standing gather part 341 is folded at a folding line 342 extending in the longitudinal direction so as to spread outward in the left-right direction, a portion of the standing gathers which is close to a free edge 343 (i.e., the free edge is one end closer to the wearer than the other end) tightly contacts with a large (wide) area of skin of the wearer.

As shown in FIG. 3, an after-mentioned side absorbent core 222 is bonded to the standing gather part 341 at a position away from the free edge 343, and the standing gather elastic member 32c is overlapped with a bonding area 344 between the standing gather part 341 and the side absorbent core 222. The folding line 342 of the standing gather part 341 is positioned between the free edge 343 and the bonding area 344, and the standing gather elastic members 32a, 32b are located between the free edge 343 and the folding line 342 of the standing gather part 341. The standing gather elastic members 32a, 32b may be located on the free edge 343 along the free edge 343 or may be located on the folding line 342 along the folding line 342, as long as the standing gather elastic members 32a, 32b are located between the free edge 343 and the folding line 342. In the present embodiment, the standing gather elastic members 32a, 32b are located on the free edge 343 and the folding line 342 of the standing gather part 341, respectively. In the case where the standing gather part 341 is not folded at the folding line 342, the standing gather elastic members 32a, 32b are located between the free edge 343 and the bonding area 344 in the standing gather part 341 (they may be located on the free edge 343 in a similar fashion to the above).

As shown in FIGS. 1 to 4, the main body part 2 has a back sheet member 23 and an absorbent sheet member 20 which is laid on (overlapped with) the back sheet member 23 (to be exact, the wearer's side surface of the back sheet member 23) and whose outer edge portion is bonded on the back sheet member 23. As shown in FIGS. 1 and 3, in a portion of the absorbent sheet member 20 which is located in rear of the middle in the longitudinal direction (i.e., the lower side in FIG. 1), an opening 25 is formed at a position to face the crotch region of the wearer. The width of the opening 25 is smaller than the width of the absorbent product 1. The back sheet member 23 has an exposed area 234 which is exposed through the opening 25 of the absorbent sheet member 20 and which is to directly face the crotch region of the wearer. At left and right sides of the opening 25, the pair of standing gather parts 341 extends in the longitudinal direction on left and right side portions of the absorbent sheet member 20.

As shown in FIG. 1, the absorbent sheet member 20 has a front absorbent core 221 located at a position to face the front side (stomach side) of the wearer when the absorbent product 1 is worn, a pair of side absorbent cores 222 located at both sides of the opening 25 in the left-right direction, and a rear absorbent core 223 located at a position to face the rear side of the wearer. The length of the front absorbent core 221 in the longitudinal direction is larger than the length of the rear absorbent core 223 in the longitudinal direction. The front absorbent core 221, the pair of side absorbent cores 222 and the rear absorbent core 223 are continuous with one another in the longitudinal direction of the absorbent sheet member 20 (the direction is also the longitudinal direction of the back sheet member 23) and formed as one unit. In the following description, the front absorbent core 221, the pair of side absorbent cores 222 and the rear absorbent core 223 are generically referred to as an “absorbent core 22”. The contour of the absorbent core 22 is drawn by thick broken lines in FIG. 1 for easy understanding of the drawing.

As shown in FIGS. 2 to 4, the absorbent sheet member 20 has a liquid-pervious core covering sheet 21 which covers an upper surface and a lower surface of each of the absorbent core 22 (i.e., these surfaces are a surface facing the wearer and a surface facing the back sheet member 23 in each of the front absorbent core 221, the side absorbent cores 222 and the rear absorbent core 223). The core covering sheet 21 has a first covering sheet 211 covering the upper surface of the absorbent core 22, and a second covering sheet 212 covering the lower surface of the absorbent core 22.

The absorbent core 22 is formed by wrapping a mixture of hydrophilic fibers such as crushed pulp fibers or cellulose fibers and granulated absorbent polymers (e.g., SAP (Super Absorbent Polymer)) in a tissue paper, a liquid-pervious nonwoven fabric or the like, and the absorbent core 22 rapidly absorbs and retains moisture which has passed through the core covering sheet 21. The tissue paper, the liquid-pervious nonwoven fabric or the like to wrap the hydrophilic fibers, is bonded to the hydrophilic fibers and the absorbent polymers with hot melt adhesive, to prevent deformation of the hydrophilic fibers and falling of the absorbent polymers (especially, falling after absorption of moisture). In the present embodiment, the absorbent core 22 includes pulp fibers and SAP.

The core covering sheet 21 is a liquid-pervious sheet material, and the core covering sheet 21 immediately catches moisture of excrement from the wearer and moves the moisture into the absorbent core 22. For example, a liquid-pervious nonwoven fabric (a point-bond nonwoven fabric, air-through nonwoven fabric, spunbond nonwoven fabric or the like) made of hydrophobic fibers (polypropylene, polyethylene, polyester, polyamide, nylon or the like) where hydrophilic treatment is performed on its surface with a surfactant, is used as the core covering sheet 21. A nonwoven fabric (for example, a spunlace nonwoven fabric) made of hydrophilic fibers such as cellulose, rayon or cotton may be used as the core covering sheet 21.

As shown in FIG. 1, a core edge 2211, which lies in the vicinity of the opening 25, of the front absorbent core 221 and a front edge 252, which lies close to the front absorbent core 221, of the opening 25 each have a concave portion depressed in the longitudinal direction at the middle in the left-right direction (i.e., the concave portion depressed toward a direction away from the rear absorbent core 223). Also a core edge 2231, which lies in the vicinity of the opening 25, of the rear absorbent core 223 and a rear edge 253, which lies close to the rear absorbent core 223, of the opening 25 each have a concave portion depressed in the longitudinal direction at the middle in the left-right direction (i.e., the concave portion depressed toward a direction away from the front absorbent core 221). The core edge 2211 of the front absorbent core 221 and the core edge 2231 of the rear absorbent core 223 may be overlapped with the front edge 252 and the rear edge 253 of the opening 25, respectively. In other words, the core edge 2211 of the front absorbent core 221 and the core edge 2231 of the rear absorbent core 223 may contact with the opening 25.

The absorbent sheet member 20 has a front absorbent core elastic member 241 which is bonded along the longitudinal direction to the front absorbent core 221 at a middle area in the left-right direction, and a rear absorbent core elastic member 243 which is bonded along the longitudinal direction to the rear absorbent core 223 at the middle area. In the absorbent sheet member 20, a contractive force in the longitudinal direction is exerted on the front absorbent core 221 by contraction of the front absorbent core elastic member 241, and a contractive force in the longitudinal direction is exerted on the rear absorbent core 223 by contraction of the rear absorbent core elastic member 243.

In the above middle area, a slit 2212 (i.e., an area in the front absorbent core 221 where pulp fibers and SAP are not provided) extending along the longitudinal direction from the core edge 2211 is formed on the front absorbent core 221, and the slit 2212 overlaps with the front absorbent core elastic member 241. Accurately, in an area where the front absorbent core 221 is provided, the front absorbent core elastic member 241 is bonded on the first covering sheet 211 and the second covering sheet 212 covering the upper surface and the lower surface of the front absorbent core 221 (see FIG. 2). In the above middle area, a slit 2232 extending along the longitudinal direction from the core edge 2231 is formed on the rear absorbent core 223, and the slit 2232 overlaps with the rear absorbent core elastic member 243. Accurately, in an area where the rear absorbent core 223 is provided, the rear absorbent core elastic member 243 is bonded on the first covering sheet 211 and the second covering sheet 212 covering the upper surface and the lower surface of the rear absorbent core 223.

In each of the front absorbent core 221 and the rear absorbent core 223, the slit 2212, 2232 is provided as a weakening line whose stiffness is lower than that of the peripheral portion. However, a weakening line where a thickness of pulp fibers or the like is thinner than that of the peripheral portion or a weakening line where a density of the pulp fibers or the like is lower than that of the peripheral portion may be provided so as to overlap with the front absorbent core elastic member 241 or the rear absorbent core elastic member 243, as a substitute for the slit 2212, 2232.

As shown in FIGS. 1 and 3, the absorbent sheet member 20 has two pairs of side absorbent core elastic members 242 (i.e., two side absorbent core elastic members 242 in each of left and right) which are located at both sides of the opening 25 in the left-right direction and which extend along the longitudinal direction. For example, a polyurethane yarn, strip-like polyurethane film, yarn-like or strip-like natural rubber, or the like is used as each of the side absorbent core elastic members 242, and in the present embodiment, a polyurethane yarn is used.

The two pairs of side absorbent core elastic members 242 are overlapped with the pair of side absorbent cores 222 in a planar view, and located on the surfaces, which face the back sheet member 23, of the side absorbent cores 222. In the present embodiment, each side absorbent core elastic member 242 is located between the side absorbent core 222 and the second covering sheet 212 of the core covering sheet 21, and bonded to the side absorbent core 222 and the second covering sheet 212 in the state where the side absorbent core elastic member 242 is stretched (i.e., the stretched side absorbent core elastic member 242 is bonded to the side absorbent core 222 and the second covering sheet 212), to exert a contractive force on the side absorbent core 222. The front portion of the side absorbent core elastic member 242 in the longitudinal direction is overlapped with the front absorbent core 221, and the rear portion is overlapped with the rear absorbent core 223.

As shown in FIGS. 2 to 4, the back sheet member 23 has a water-repellent or liquid-impervious outer covering sheet 231, a very thin absorbent sheet 233 provided on the outer covering sheet 231 (i.e., the wearer's side surface of the outer covering sheet 231), and a hydrophilic sheet 232 which is laminated on the outer covering sheet 231 and the absorbent sheet 233. In FIGS. 2 to 4, the absorbent sheet 233 is drawn by a thick solid line for the convenience of illustration.

As the outer covering sheet 231, used is a water-repellent or liquid-impervious nonwoven fabric (for example, a spunbond nonwoven fabric, meltblown nonwoven fabric or SMS nonwoven fabric) made of hydrophobic fibers (for example, polypropylene, polyethylene, polyester, polyamide, nylon), a water-repellent or liquid-impervious plastic film, or a laminated sheet of the nonwoven fabric and the plastic film. The outer covering sheet 231 prevents moisture of excrement or the like which has come to the back sheet member 23, from leaking out to the outer side of the main body part 2. In a case where a plastic film is used for the outer covering sheet 231, it is preferable that a plastic film with permeability (breathability) is used, from the view point of preventing sweatiness in the absorbent product 1 and providing comfortable feeling to the wearer.

The absorbent sheet 233 has two sheets and a high absorbent resin layer provided between the two sheets, and the high absorbent resin layer is formed by fixing granulated absorbent polymers such as SAP on the two sheets with hot melt adhesive. As the two sheets, a nonwoven fabric made of hydrophilic fibers, a nonwoven fabric made of hydrophobic fibers after hydrophilic treatment, tissue or the like is used. The high absorbent resin layer (i.e., granulated absorbent polymers) is located between the above two sheets and laid in form of stripes extending in the longitudinal direction. In other words, areas where absorbent polymers do not exist are provided between high absorbent resin layer elements (corresponding to the stripes) extending in the longitudinal direction. Each of the high absorbent resin layer elements is sealed by bonding the two sheets at the areas where absorbent polymers do not exist.

As a substitute for the above absorbent sheet 233, a high absorbent resin layer may be directly formed on the outer covering sheet 231 by spreading granulated absorbent polymers such as SAP on the outer covering sheet 231 and by bonding them on the outer covering sheet 231 with hot melt adhesive or the like. A nonwoven fabric (for example, a spunlace nonwoven fabric) made of hydrophilic fibers such as cellulose, rayon or cotton, or a liquid-pervious nonwoven fabric made of hydrophobic fibers (polypropylene, polyethylene, polyester, polyamide, nylon or the like) where hydrophilic treatment is performed on its surface with a surfactant, is preferably used as the hydrophilic sheet 232.

In the absorbent product 1, as described above, the outer edge portion of the absorbent sheet member 20 is bonded on the back sheet member 23, and the surface of the absorbent core 22 which faces the back sheet member 23 is not bonded to the back sheet member 23. Therefore, as shown in FIGS. 2 and 4, a front pocket 26 elongating (spreading) toward front side of the longitudinal direction from the opening 25 is formed between the front absorbent core 221 and the back sheet member 23 (i.e., between a portion of the absorbent sheet member 20 corresponding to the front absorbent core 221, and the back sheet member 23), and as shown in FIG. 3, a pair of side pockets 28 deepening (spreading) in the left-right direction from the opening 25 is formed between the pair of side absorbent cores 222 and the back sheet member 23. As shown in FIG. 4, a rear pocket 27 elongating (spreading) toward rear side of the longitudinal direction from the opening 25 is formed between the rear absorbent core 223 and the back sheet member 23 in a similar fashion to the front pocket 26. The pair of side pockets 28 is continuous with the front pocket 26 and the rear pocket 27.

In the absorbent product 1 shown in FIG. 1, excrement such as loose stool excreted from the wearer passes through the opening 25 to reach the back sheet member 23, and the excrement is received by the exposed area 234 of the back sheet member 23. The excrement on the exposed area 234 moves into internal spaces of the front pocket 26, the side pockets 28 and the rear pocket 27 to be collected. In the present embodiment, the opening 25 is provided at rear side of the middle of the absorbent sheet member 20 in the longitudinal direction, and the front absorbent core 221 is longer than the rear absorbent core 223 as described above. Therefore, the length of the front pocket 26 in the longitudinal direction is larger than that of the rear pocket 27, and the front pocket 26 becomes a main pocket to mainly collect excrement.

In the absorbent product 1, as described above, the pair of side absorbent cores 222 is bonded to the inner surfaces of the pair of standing gather parts 341 (i.e., the surfaces face the back sheet member 23) through the core covering sheet 21 (see FIG. 3). Thus, by contraction of one pair of standing gather elastic members 32a and another pair of standing gather elastic members 32b, the standing gather parts 341 are lifted (stood up) significantly, and the side absorbent cores 222 become far away from the back sheet member 23. Therefore, with respect to the vicinity of the opening 25, a distance in a thickness direction between the absorbent sheet member 20 and the back sheet member 23 can be kept large. As the result, in the absorbent product 1, since excrement can be securely collected in the front pocket 26, the side pockets 28 and the rear pocket 27 to be kept therein, the excrement is prevented from attaching over a large area of skin of the wearer.

As shown in FIG. 3, side wall parts 281 of the side pockets 28 have structure where the side absorbent cores 222 are bonded to the inner sides (inner surfaces) of the side sheets 3 and the outer covering sheet 231, to thereby increase stiffness of the side wall parts 281 of the side pockets 28. It is therefore possible to suppress deformation of the side wall parts 281 due to a weight of excrement collected in the side pockets 28 and to suppress leakage of urine or the like from side portions of the absorbent product 1 (so-called lateral leakage) due to the deformation of the side wall parts 281. Furthermore, since the side absorbent cores 222 contain pulp fibers, the stiffness of the side wall parts 281 is further increased. In left and right sides of the opening 25, since the inner surfaces of the side wall parts 281 have absorbent property by the side absorbent cores 222, lateral leakage of urine or the like can be further suppressed.

As above, although the absorbent product 1 covers only a relatively-small area of skin of the wearer, the absorbent product 1 can securely collect excrement and hold it. Thus, the absorbent product 1 is especially suitable as an auxiliary absorbent pad which is to be attached on the inside of an exterior product such as disposable diaper and which can be changed easily.

In the meantime, since a wearer of the absorbent product 1 is normally in a supine position, lateral position or sitting position or the like, body pressure is hardly applied on the front side of the wearer. In the absorbent product 1, as described above, the front pocket 26 formed between the front absorbent core 221 located to the front side of the wearer and the back sheet member 23 is made to be a main pocket to mainly collect excrement. Thus, the absorbent product 1 is especially suitable for collecting excrement from a wearer who takes various body positions such as a supine position, lateral position or sitting position.

In the absorbent product 1, since the two pairs of side absorbent core elastic members 242 extending in the longitudinal direction are located to overlap with the pair of side absorbent cores 222, the stiffness of the side wall parts 281 of the side pockets 28 can be further increased to further suppress deformation of the side wall parts 281. In addition, since these pairs of side absorbent core elastic members 242 exert a contractive force on the pair of side absorbent cores 222, the side absorbent cores 222 can be further away from the back sheet member 23. Furthermore, since the side absorbent core elastic members 242 are located on the surfaces, facing the back sheet member 23, of the side absorbent cores 222, the side absorbent cores 222 can be further away from the back sheet member 23. Only one pair of side absorbent core elastic members 242 may be provided to the absorbent sheet member 20. In this case, it is preferable that the pair of side absorbent core elastic members 242 is located in the vicinity of the opening 25 with respect to the left-right direction (i.e., each side absorbent core elastic member 242 is located so as to be closer to the opening 25 than the middle portion of the side absorbent core 222 in the left-right direction). Therefore, portions, in the vicinity of the opening 25, of the side pockets 28 can be far away from the back sheet member 23.

In the pair of standing gather parts 341, as described above, since the pair of standing gather elastic members 32c extending in the longitudinal direction is located to overlap with the bonding areas 344 between the pair of standing gather parts 341 and the pair of side absorbent cores 222, the stiffness of the side wall parts 281 of the side pockets 28 can be further increased to further suppress deformation of the side wall parts 281. By a contractive force of these standing gather elastic members 32c, the pair of standing gather parts 341 is made to stand up significantly, and the side absorbent cores 222 can be far away from the back sheet member 23.

In the absorbent product 1, the core edge 2211 of the front absorbent core 221 and the core edge 2231 of the rear absorbent core 223 have the concave portions depressed in the longitudinal direction at the middle in the left-right direction. Therefore, while it is ensured that a length of the opening 25 in the longitudinal direction is large at the middle in the left-right direction, lengths, in the longitudinal direction, of the side wall parts 281 of the side pockets 28 can be shortened. As the result, the stiffness of the side wall parts 281 can be further increased.

Since the front absorbent core elastic member 241 and the rear absorbent core elastic member 243 extending in the longitudinal direction are bonded to the middle area, in the left-right direction, of the front absorbent core 221 and the rear absorbent core 223 to exert contractive forces on the front absorbent core 221 and the rear absorbent core 223, the middle area of the front absorbent core 221 and the rear absorbent core 223 becomes far away from the back sheet member 23. Furthermore, since the slits 2212, 2232 overlapping with the front absorbent core elastic member 241 and the rear absorbent core elastic member 243, respectively, are provided on the middle area, in the left-right direction, of the front absorbent core 221 and the rear absorbent core 223, the front absorbent core 221 and the rear absorbent core 223 easily deform into a form protruding toward the wearer. Therefore, the middle area of the front absorbent core 221 and the rear absorbent core 223 becomes far away from the back sheet member 23. As the result, collective capacity of the front pocket 26 and the rear pocket 27 can be increased.

In the absorbent product 1, as described above, the pair of side absorbent cores 222 and the front absorbent core 221 are continuous with each other in the longitudinal direction and formed as one unit. Thus, when the side absorbent cores 222 become far away from the back sheet member 23 by rising (standing up) of the standing gather parts 341, the portion of the front absorbent core 221 which lies in the vicinity of the opening 25 can become also far away from the back sheet member 23. In addition, the pair of side absorbent cores 222 and the rear absorbent core 223 are continuous with each other in the longitudinal direction and formed as one unit. Thus, when the side absorbent cores 222 become far away from the back sheet member 23 by rising of the standing gather parts 341, the portion of the rear absorbent core 223 which lies in the vicinity of the opening 25 can become also far away from the back sheet member 23. As the result, the collective capacity of the front pocket 26 and the rear pocket 27 can be further increased.

Though the preferred embodiments of the present invention have been discussed above, the present invention is not limited to the above-discussed preferred embodiments, but allows various variations.

For example, the absorbent core 22 doesn't always have to contain pulp fibers, and a member where SAP is directly bonded on a liquid-pervious nonwoven fabric, which is relatively thick and which has high stiffness or a plastic film, with hot melt adhesive or the like, may be used as the absorbent core 22.

In the absorbent product 1, in the case where portions, in the vicinity of the opening 25, of the front pocket 26 and the rear pocket 27 can be significantly far away from the back sheet member 23, there may be a case where the front absorbent core 221, the pair of side absorbent cores 222 and the rear absorbent core 223 are formed as separate members, they are located adjacent to one another so as to form the opening 25 (i.e., located around an opening of the core covering sheet 21) and covered with the core covering sheet 21, for example. In the case where the side absorbent cores 222 can be significantly far away from the back sheet member 23, for example, at the upper side (wearer's side) of the side absorbent core 222, each side absorbent core elastic member 242 may be bonded between the side absorbent core 222 and the first covering sheet 211 of the core covering sheet 21.

In the absorbent product 1, in order to increase the collective capacity of excrement, it is preferable that the front pocket 26, the side pockets 28 and the rear pocket 27 are provided between the absorbent sheet member 20 and the back sheet member 23. However, in the case where the collective capacity of excrement is ensured sufficiently, there may be a case where only the front pocket 26 which is a main pocket and the pair of side pockets 28 are provided between the absorbent sheet member 20 and the back sheet member 23, and the rear pocket 27 is omitted. In the case where the rear pocket 27 is not provided, an almost whole surface of a portion, which is in rear of the opening 25, of the absorbent sheet member 20 is bonded to the back sheet member 23. In this case, the rear absorbent core 223 may be omitted from the absorbent sheet member 20.

On the other hand, when the absorbent product 1 is used for a wearer who often takes a body position such as a prone position, where body pressure is easily applied to the front side of the wearer, the rear pocket 27 formed between the rear absorbent core 223 and the back sheet member 23 may be the main pocket. In the case where the rear pocket 27 is the main pocket, the front pocket 26 and the front absorbent core 221 may be omitted.

In other words, out of the absorbent core 22, a portion which is located at a position to face one of the front side and the rear side of the wearer and which forms the main pocket together with the back sheet member 23 is treated as a first absorbent core, the pair of side absorbent cores 222 which forms the pair of side pockets 28 together with the back sheet member 23 is treated as a pair of second absorbent cores, a portion which is located at a position to face the other of the front side and the rear side of the wearer and which forms another pocket together with the back sheet member 23, the another pocket being other than the main pocket and the side pockets 28 and being applied with body pressure, is treated as a third absorbent core, and in this case, the third absorbent core and the pocket formed between the third absorbent core and the back sheet member 23 may be omitted in the absorbent product 1.

In the absorbent sheet member 20, the core covering sheet 21 may be one sheet member. In this case, a portion of the sheet member covering the wearer's side surface of the absorbent core 22 is equivalent to the first covering sheet 211, and a portion covering the other surface, facing the back sheet member 23, of the absorbent core 22 is equivalent to the second covering sheet 212. The core covering sheet 21 has only to cover at least the wearer's side surface of the absorbent core 22.

The back sheet member 23 has only to have at least the water-repellent or liquid-impervious outer covering sheet 231, and any one or both of the hydrophilic sheet 232 and the absorbent sheet 233 may be omitted.

Structure of the above absorbent product may be applied to, for example a pants-type (i.e., pull-up type) disposable diaper which has a waist opening at an upper end and a pair of leg openings on a lower part, an open-type (tape-type) disposable diaper where a portion located on the front side of a wearer and a portion located on the rear side are fastened around the waistline of the wearer in wearing the disposable diaper, other than the auxiliary absorbent pad to be attached at the inside of an exterior product.

While the invention has been shown and described in detail, the foregoing description is in all aspects illustrative and not restrictive. It is therefore understood that numerous modifications and variations can be devised without departing from the scope of the invention.

### REFERENCE SIGNS LIST

1 absorbent product

20 absorbent sheet member

21 core covering sheet

23 back sheet member

25 opening

26 front pocket

27 rear pocket

28 side pocket

32a to 32c standing gather elastic member

221 front absorbent core

222 side absorbent core

223 rear absorbent core

241 front absorbent core elastic member

242 side absorbent core elastic member

243 rear absorbent core elastic member

341 standing gather part

342 folding line

343 free edge

344 bonding area

2211, 2231 core edge

2212, 2232 slit

1. An absorbent product for receiving excrement from a wearer, comprising:
a back sheet member;
an absorbent sheet member which is laid on said back sheet member and whose outer edge portion is bonded on said back sheet member, said absorbent sheet member having an opening at a position to face a crotch region of a wearer, said back sheet member being exposed through said opening; and
a pair of standing gather parts lying on left and right side portions of said absorbent sheet member and extending in a longitudinal direction, a pair of standing gather elastic members along said longitudinal direction being bonded to said pair of standing gather parts, respectively; wherein
said absorbent sheet member comprises:
a first absorbent core located at a position to face one of a front side and a rear side of said wearer;
a pair of second absorbent cores located at both sides of said opening in a left-right direction; and
a liquid-pervious core covering sheet which covers said first absorbent core and said pair of second absorbent cores;
a main pocket elongating in said longitudinal direction from said opening is formed between said first absorbent core and said back sheet member, a pair of side pockets deepening in said left-right direction from said opening is formed between said pair of second absorbent cores and said back sheet member, and said pair of second absorbent cores is bonded to said pair of standing gather parts through said core covering sheet.

2. The absorbent product according to claim 1, wherein
said absorbent sheet member further comprises a pair of second absorbent core elastic members which is located along said longitudinal direction on surfaces, facing said back sheet member, of said pair of second absorbent cores, said pair of second absorbent core elastic members exerting a contractive force on said pair of second absorbent cores.

3. The absorbent product according to claim 1, wherein
free edges of said pair of standing gather parts are away from bonding areas between said pair of standing gather parts and said pair of second absorbent cores, said pair of standing gather elastic members is located between said free edges and said bonding areas in said pair of standing gather parts, and
another pair of standing gather elastic members is bonded to said pair of standing gather parts while overlapping with said bonding areas along said longitudinal direction.

4. The absorbent product according to claim 1, wherein
free edges of said pair of standing gather parts are away from bonding areas between said pair of standing gather parts and said pair of second absorbent cores, said pair of standing gather parts is folded outward in said left-right direction at folding lines extending in said longitudinal direction and lying between said free edges and said bonding areas, and said pair of standing gather elastic members is located between said free edges and said folding lines in said pair of standing gather parts.

5. The absorbent product according to claim 1, wherein
a core edge, which lies in the vicinity of said opening or contacts with said opening, of said first absorbent core has a concave portion depressed in said longitudinal direction at the middle in said left-right direction.

6. The absorbent product according to claim 1, wherein
said absorbent sheet member further comprises a first absorbent core elastic member which is bonded along said longitudinal direction to said first absorbent core at a middle area in said left-right direction, said first absorbent core elastic member exerting a contractive force on said first absorbent core.

7. The absorbent product according to claim 6, wherein
said first absorbent core has a weakening line which overlaps with said first absorbent core elastic member at said middle area.

8. The absorbent product according to claim 1, wherein
said first absorbent core and said pair of second absorbent cores are continuous with each other in said longitudinal direction and formed as one unit.

9. The absorbent product according to claim 1, wherein
said first absorbent core is a front absorbent core located at a position to face a front side of said wearer.

10. The absorbent product according to claim 1, wherein
said absorbent sheet member further comprises a third absorbent core located at a position to face the other of a front side and a rear side of said wearer, and another pocket is formed between said third absorbent core and said back sheet member.

11. The absorbent product according to claim 1, wherein
said absorbent product is an auxiliary absorbent pad to be attached on the inside of an exterior product worn by a wearer.

12. The absorbent product according to claim 2, wherein
said absorbent product is an auxiliary absorbent pad to be attached on the inside of an exterior product worn by a wearer.

13. The absorbent product according to claim 3, wherein
said absorbent product is an auxiliary absorbent pad to be attached on the inside of an exterior product worn by a wearer.

14. The absorbent product according to claim 4, wherein
said absorbent product is an auxiliary absorbent pad to be attached on the inside of an exterior product worn by a wearer.

15. The absorbent product according to claim 5, wherein
said absorbent product is an auxiliary absorbent pad to be attached on the inside of an exterior product worn by a wearer.

16. The absorbent product according to claim 6, wherein
said absorbent product is an auxiliary absorbent pad to be attached on the inside of an exterior product worn by a wearer.

17. The absorbent product according to claim 7, wherein
said absorbent product is an auxiliary absorbent pad to be attached on the inside of an exterior product worn by a wearer.

18. The absorbent product according to claim 8, wherein
said absorbent product is an auxiliary absorbent pad to be attached on the inside of an exterior product worn by a wearer.

19. The absorbent product according to claim 9, wherein
said absorbent product is an auxiliary absorbent pad to be attached on the inside of an exterior product worn by a wearer.

20. The absorbent product according to claim 10, wherein
said absorbent product is an auxiliary absorbent pad to be attached on the inside of an exterior product worn by a wearer.
